(19) United States
(12) Patent Application Publication
(10) Pub. No.: US 2009/0030287 A1
(43) Pub. Date: Jan. 29, 2009

(54) INCENTED RESPONSE ASSESSMENT AT A POINT OF TRANSACTION

(75) Inventors: Anantha Pradeep, Berkeley, CA (US); Robert T. Knight, Berkeley, CA (US); Ramachandran Gurumoorthy, Berkeley, CA (US); Ratnakar Dev, Berkeley, CA (US)

Correspondence Address: Weaver Austin Villeneuve & Sampson LLP, P.O. BOX 70250, OAKLAND, CA, 94612-0250, US

(73) Assignee: NEUROFOCUS INC., Berkeley, CA (US)

(21) Appl. No.: 12/135,069

(22) Filed: Jun. 6, 2008

Related U.S. Application Data
(60) Provisional application No. 60/942,311, filed on Jun. 6, 2007.

(51) Int. Cl. A61B 5/00

(52) U.S. Cl. 600/300

(57) ABSTRACT

Subjects exposed to stimulus materials such stimulus associated with products and services are provided with incentives to provide response assessments at a point of transaction. The point of transaction has a time and/or location near the point of exposure to the stimulus materials and response collection. In some examples, the point of transaction is associated with a product request, information request, service request, product delivery, information download, service fulfillment, etc. Response data is collected at the point of transaction to more accurately assess user responses to stimulus materials.

### CROSS REFERENCE TO RELATED APPLICATIONS

This application claims priority to Provisional Patent Application 60/942,311 (Docket No. 2007NF10) titled Incented Response Assessment Device At Point Of Transaction Or Point Of Service, by Anantha Pradeep, Robert T. Knight, Ramachandran Gurumoorthy, and filed on Jun. 6, 2007.

### TECHNICAL FIELD

The present disclosure relates to an incented response assessment.

### DESCRIPTION OF RELATED ART

Conventional systems for performing response assessment typically measure responses and monitor stimulus provided to particular subjects in an inefficient and ineffective manner. Mechanisms for performing response assessment are limited, and often rely on demographic information, statistical, user behavioral, and survey based response collection.

Consequently, it is desirable to provide improved methods and apparatus for performing response assessment.

### BRIEF DESCRIPTION OF THE DRAWINGS

The disclosure may best be understood by reference to the following description taken in conjunction with the accompanying drawings, which illustrate particular example embodiments.

FIG. 1 illustrates one example of a system for performing response assessment.

FIG. 2 illustrates examples of stimulus attributes that can be included in a stimulus and audience attributes repository.

FIG. 3 illustrates examples of data models that can be used with the response assessment system.

FIG. 4 illustrates one example of a query that can be used with the response assessment system.

FIG. 5 illustrates one example of a report generated using the response assessment system.

FIG. 6 illustrates one example of a technique for performing response assessment.

FIG. 7 provides one example of a system that can be used to implement one or more mechanisms.

### DESCRIPTION OF PARTICULAR EMBODIMENTS

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques and mechanisms of the present invention will be described in the context of particular types of transactions and points of transactions. However, it should be noted that the techniques and mechanisms of the present invention apply to a variety of transactions and variations. Furthermore, it should be noted that various mechanisms and techniques can be applied to a variety of stimuli. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

### Overview

Subjects exposed to stimulus materials such stimulus associated with products and services are provided with incentives to provide response assessments at a point of transaction. The point of transaction has a time and/or location near the point of exposure to the stimulus materials and response collection. In some examples, the point of transaction is associated with a product request, information request, service request, product delivery, information download, service fulfillment, etc. Response data is collected at the point of transaction to more accurately assess user responses to stimulus materials.

### Example Embodiments

Conventional response assessment mechanisms merely track stimulus being experienced and rely on behavior and survey based data collected from subjects exposed to materials. The survey based instruments typically measure the response at points not tied to a particular transaction such as a product transaction or a service transaction, and typically provide incentives separated from a point of transaction.

Conventional response measurement devices also do not integrate the audience psychographic, neurographic, or demographic profiles in the response assessment and also do not integrate attributes and meta-information about the stimulus presented in assessing the response. Many response measurement devices also do not provide mechanisms that can be run by parties participating in the transaction such as product and service providers or third party aggregators.

Typical response assessment mechanisms also do not provide incentives tied to points of transaction. Consequently, the techniques of the present invention provide an incented response assessment mechanism that measures and tracks response to stimulus at a point of transaction. According to various embodiments, the incented response assessment mechanism includes a transaction identifier that automatically or semi-automatically identifies a transaction such as a request, delivery, of fulfillment. In particular embodiments, the incented response assessment mechanism also includes an incented response request device that presents an incentive typically tied to the transaction and requests a user response to identified stimulus. A stimulus presentation and response collection device may also be included to present identified stimulus and collect and store user responses. A response analyzer processes data collected using multiple techniques to elicit insights and assessments.

According to various embodiments, the incented response assessment device at a point of transaction provides a mechanism to integrate the response to the stimulus with stimulus attributes including meta-information and integrates audience attributes like demographic, psychographic, and neurographic profiles into the response assessment.

The incented response assessment device may also store and track multiple transactions by the same user and use this information to modify response requests and/or incentives as well as points of introduction of response collection. The incented response assessment can be performed as part of a transaction request or fulfillment process, or may be implemented as a third party service. In some examples, the incented response assessment system also uses neuro-response measurements such as central nervous system, autonomic nervous system, and effector measurements that may be taken at a point of transaction or at another time to improve response assessment.

Some examples of central nervous system measurement mechanisms include Functional Magnetic Resonance Imaging (fMRI) and Electroencephalography (EEG). fMRI measures blood oxygenation in the brain that correlates with increased neural activity. However, current implementations of fMRI have poor temporal resolution of few seconds. EEG measures electrical activity associated with post synaptic currents occurring in the milliseconds range. Subcranial EEG can measure electrical activity with the most accuracy, as the bone and dermal layers weaken transmission of a wide range of frequencies. Nonetheless, surface EEG provides a wealth of electrophysiological information if analyzed properly.

Autonomic nervous system measurement mechanisms include Galvanic Skin Response (GSR), Electrocardiograms (EKG), pupillary dilation, etc. Effector measurement mechanisms include Electrooculography (EOG), eye tracking, facial emotion encoding, reaction time etc.

According to various embodiments, the techniques and mechanisms of the present invention intelligently blend multiple modes and manifestations of precognitive neural signatures with cognitive neural signatures and post cognitive neurophysiological manifestations to more accurately allow assessment of response to stimulus material. In some examples, autonomic nervous system measures are themselves used to validate central nervous system measures. Effector and behavior responses are blended and combined with other measures. According to various embodiments, central nervous system, autonomic nervous system, and effector system measurements are aggregated into a measurement that allows definitive evaluation of response data of stimulus material.

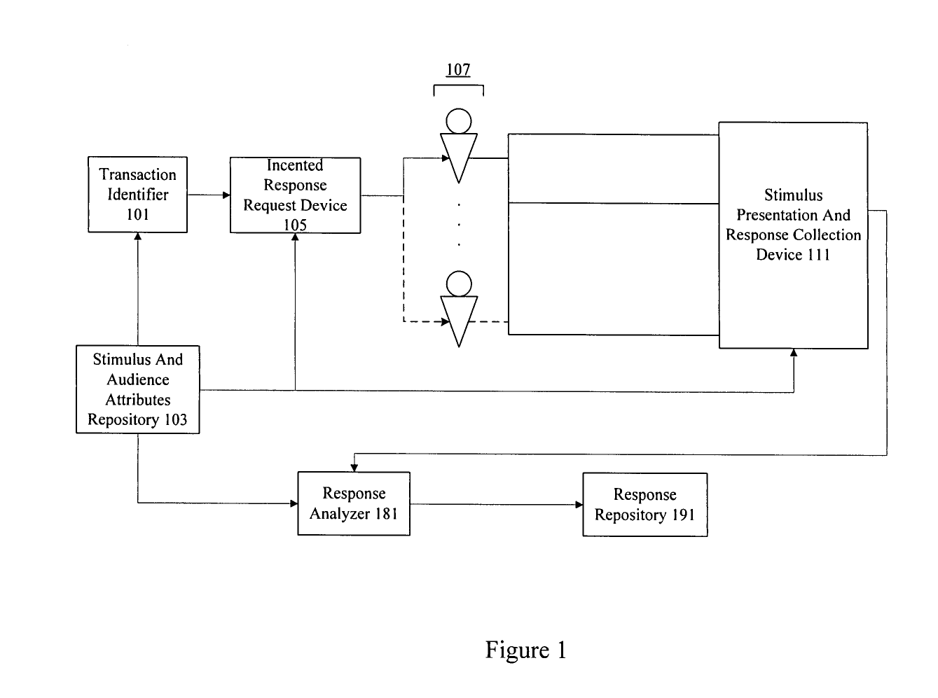

FIG. 1 illustrates one example of a system for performing response assessment using central nervous system, autonomic nervous system, and effector measures. According to various embodiments, a transaction identifier 101 is provided to automatically or semi-automatically identify a transaction such as a service, information, or product request. In particular embodiments, the transaction may be an Internet, phone, or web based transaction or service request. In other examples, the transaction may be a human assisted retail transaction or business transaction. In still other examples, the transaction may be an automated transaction such as an Automated Teller Machine (ATM), vending machine, or remote purchase transaction. The transaction may also be a payment transaction such as a credit card, ATM card, or electronic payment transaction associated with a product or service purchase, donation, or money transfer. A variety of transactions may be identified and stimulus associated with the variety of transactions can be assessed at the point of transaction.

According to various embodiments, stimulus is obtained from a stimulus and audience attributes repository 103. A stimulus and audience attributes repository 103 provides information on the stimulus material being presented to an audience as well as information on the audience itself. According to various embodiments, stimulus attributes include properties of the stimulus materials as well as purposes, presentation attributes, report generation attributes, etc. In particular embodiments, stimulus attributes include time span, channel, rating, media, type, etc. Audience attributes include demographic, psychographic, and neurographic profiles of subjects in response assessment. Other attributes such as purpose attributes and presentation attributes may also be included. Purpose attributes include aspiration and objects of the stimulus including excitement, memory retention, associations, etc. Presentation attributes include audio, video, imagery, and message needed for enhancement or avoidance. Other attributes may or may not also be included in the stimulus and audience attributes repository or some other repository.

According to various embodiments, the transaction identifier 101 and the stimulus and audience attributes repository 103 provide information to an incented response request device 105. In particular embodiments, the incented response request device 105 provides an incentive, typically tied to the particular transaction, and requests the user response to identified stimuli. The stimuli may be associated with the transaction or may be provided by the incented response request device 105 itself. According to various embodiments, the incented response request device 105 is implemented using a web page, a human request at a point of service, or multiple screens at an ATM or credit card reader. The incentive provide may be a discount, coupon, credit, reward, and can be tied to the value of the transaction. Incentives may also be applied directly to user accounts.

According to various embodiments, incentives are selected using a stimulus and audience attributes repository 103 to intelligently select incentives that suit a particular user and a particular situation. Incentives may relate to services or products included in the transaction or may be tied to particular user interests. In particular embodiments, incentives may be selected by service and product providers with or without the use of user profile information. The incented response request device 105 may also include mechanisms to identify and track usage history, user/group profiles, transaction characteristics, and use this information to provide more effective incentives to initiate response requests as well as to select more effective times at which incented response requests should be introduced. Attributes such as behavioral, neurophysiological, and neuro-behavioral attributes of the transaction can also be used to select incentives and times at which incented response requests should be introduced.

The incented response request device 105 provides incentives to multiple subjects 107. According to various embodiments, the multiple subjects 107 are customers, clients, users involved in transactions in a variety of contexts. In some examples, multiple subjects 107 include network users that make requests for products, services, or information. Multiple subjects 107 may also include customers reviewing a product at a kiosk or making a purchase at a vendor. The multiple subjects 107 may be provided with incentives and a response request at any point of transaction. The multiple subjects 107 may also be provided with additional stimulus that may or may not be associated with the transaction.

In particular embodiments, the multiple subjects 107 have profiles maintained by a response assessment system. In some examples, the profiles are tied to particular credit cards, ATM cards, and user identifiers. According to various embodiments, stimulus presentation and response collection device 111 obtains responses from subjects and maps the responses to particular stimulus material associated with subject transactions.

According to various embodiments, the subjects are connected to the stimulus presentation and response collection device 111. In particular embodiments, the stimulus presented includes audio/visual/tactile/olfactory and other sensory stimuli. These could be used to elicit user assessments of the transaction such as a product or service being requested, provided, or fulfilled and may involve attributes of products tied to the transaction. In particular embodiments, presentation of the stimuli is independent of the transaction or service.

The stimuli could be presented individually to users (1 system) or simultaneously to a group of users (1+N system). According to various embodiments, the stimulus presentation and response collection device 111 collects attributes of the stimuli and its presentation such as the time and region of presentation, the duration of the presentation and the response, the creator/sponsor/provider of the stimuli, user response attributes, etc. The stimulus material may be a media clip, a commercial, pages of text, a brand image, a performance, a magazine advertisement, a movie, an audio presentation, particular tastes, smells, textures and/or sounds. The stimuli can involve a variety of senses and occur with or without human supervision. Continuous and discrete modes are supported. According to various embodiments, the stimulus presentation and response collection device 111 also has protocol generation capability to allow intelligent customization of stimuli provided to multiple subjects. The stimulus presentation and response collection device 111 can be based on push, pull or a push/pull mechanism interacting with the user.

In particular embodiments, the stimulus presentation and response collection device 111 also includes data input mechanisms such as keypads, touchpads, keyboards, mice, voice recognition devices, forms, buttons, switches, etc. that allow a subject to provide response information. The stimulus presentation and response collection device 111 may operate automatically or may be enhanced with human interaction. Although the stimulus presentation and response collection device 111 may not include neuro-response measurement mechanisms, it should be recognized that neuro-response measurement mechanisms can also be used.

According to various embodiments, the stimulus presentation and response collection device 111 may also include a variety of neuro-response measurement mechanisms including behavioral, statistical, survey, and neurophysiological measurements systems such as EEG, EOG, GSR, EKG, pupillary dilation, eye tracking, facial emotion encoding, and reaction time devices, etc. According to various embodiments, the statistical and survey mechanisms includes non-linear, geometric, and spiral rating mechanisms. According to various embodiments, neuro-response data includes central nervous system, autonomic nervous system, and effector data. In particular embodiments, the stimulus presentation and response collection device 111 include EEG, EOG, and GSR. In some instances, only a single data collection device is used. Data collection may proceed with or without human supervision.

In one particular embodiment, the response assessment system includes EEG measurements made using scalp level electrodes, EOG measurements made using shielded electrodes to track eye data, GSR measurements performed using a differential measurement system, a facial muscular measurement through shielded electrodes placed at specific locations on the face, and a facial affect graphic and video analyzer adaptively derived for each individual.

According to various embodiments, the response assessment system also includes a data cleanser device. In particular embodiments, the data cleanser device filters the collected data to remove noise, artifacts, and other irrelevant data using fixed and adaptive filtering, weighted averaging, advanced component extraction, etc.

The stimulus presentation and response collection device 111 and the stimulus and audience attributes repository 103 pass data to the response analyzer 181. The response analyzer 181 uses a variety of mechanisms to analyze underlying data in the system to determine response characteristics of stimulus material.

According to various embodiments, the response analyzer customizes and extracts the independent behavioral, statistical, survey, and neuro-physiological parameters for each individual, and blends the estimates to elicit an enhanced response to the presented stimulus material. In particular embodiments, the response analyzer 181 aggregates the response measures across subjects in a dataset. The response measures can be used to identify and build user and user group profiles. The identified profiles could be integrated and correlated with the user responses to elicit further insights.

According to various embodiments, behavioral, statistical, survey, and neuro-physiological signatures are measured using time domain analyses and frequency domain analyses. Such analyses use parameters that are common across individuals as well as parameters that are unique to each individual. The analyses could also include statistical parameter extraction and fuzzy logic based attribute estimation from both the time and frequency components of the synthesized response.

In some examples, statistical parameters used in a blended effectiveness estimate include evaluations of skew, peaks, first and second moments, population distribution, as well as fuzzy estimates of attention, emotional engagement and memory retention responses.

According to various embodiments, the response analyzer 181 may include an intra-modality response synthesizer and a cross-modality response synthesizer. In particular embodiments, the intra-modality response synthesizer is configured to customize and extract the independent behavioral, statistical, survey, and neurophysiological parameters for each individual in each modality and blend the estimates within a modality analytically to elicit an enhanced response to the presented stimuli. In particular embodiments, the intra-modality response synthesizer also aggregates data from different subjects in a dataset.

According to various embodiments, the cross-modality response synthesizer or fusion device blends different intra-modality responses, including raw signals and signals output. The combination of signals enhances the measures of effectiveness within a modality. The cross-modality response fusion device can also aggregate data from different subjects in a dataset.

According to various embodiments, the response analyzer 181 also includes a composite enhanced effectiveness estimator (CEEE) that combines the enhanced responses and estimates from each modality to provide a blended estimate of the effectiveness. In particular embodiments, blended estimates are provided for each exposure of a subject to stimulus materials. The blended estimates are evaluated over time to determine response characteristics. According to various embodiments, numerical values are assigned to each blended estimate. The numerical values may correspond to the intensity of neuro-response measurements, the significance of peaks, the change between peaks, etc. Higher numerical values may correspond to higher significance in response intensity. Lower numerical values may correspond to lower significance or even insignificant response activity. In other examples, multiple values are assigned to each blended estimate. In still other examples, blended estimates of response significance are graphically represented to show changes after repeated exposure.

According to various embodiments, the response analyzer 181 provides analyzed and enhanced response data to a response repository 191. According to various embodiments, the response repository 191 maintains analyzed and enhanced response data for retrieval, processing, report generation, etc. In particular embodiments, the response repository 183 includes mechanisms for the compression and encryption of data for secure storage and communication.

As with a variety of the components in the response assessment system, the response repository can be co-located with the rest of the system and the user, or could be implemented in a remote location. It could also be optionally separated into an assessment repository system that could be centralized or distributed at the provider or providers of the stimulus material. In other examples, the response repository is housed at the facilities of a third party service provider accessible by stimulus material providers and/or users.

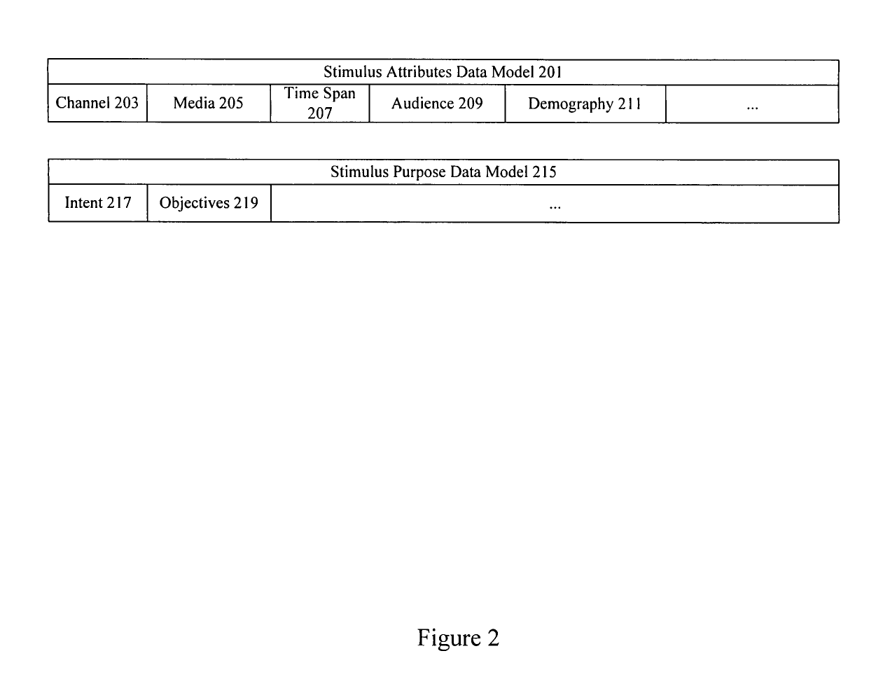

FIG. 2 illustrates examples of data models that may be provided with a stimulus and audience attributes repository. According to various embodiments, a stimulus attributes data model 201 includes a channel 203, media type 205, time span 207, audience 209, and demographic information 211. A stimulus purpose data model 215 may include intents 217 and objectives 219.

According to various embodiments, intent and objectives may include memory retention of a brand name, association of a product with a particular feeling, excitement level for a particular service, etc. The attributes may be useful in providing targeted stimulus materials to multiple subjects and tracking and evaluating the effectiveness of the stimulus materials.

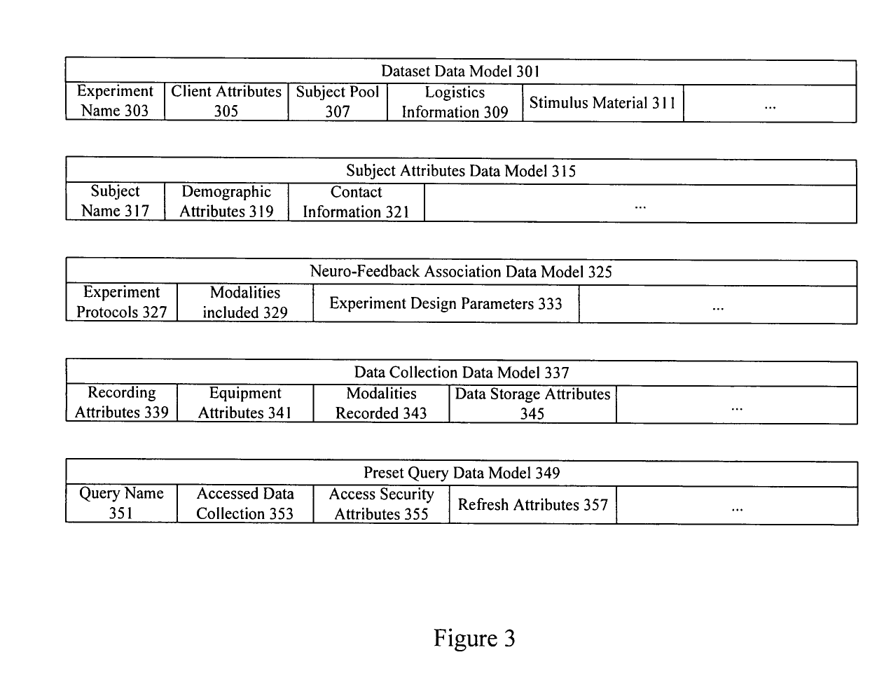

FIG. 3 illustrates examples of data models that can be used for storage of information associated with tracking and measurement of responses. According to various embodiments, a dataset data model 301 includes an experiment name 303 and/or identifier, client attributes 305, a subject pool 307, logistics information 309 such as the location, date, and time of testing, and stimulus material 311 including stimulus material attributes.

In particular embodiments, a subject attribute data model 315 includes a subject name 317 and/or identifier, contact information 321, and demographic attributes 319 that may be useful for review of behavioral, statistical, psychographic, survey, and neuro-response data. Some examples of pertinent demographic attributes include marriage status, employment status, occupation, household income, household size and composition, ethnicity, geographic location, sex, race. Other fields that may be included in data model 315 include shopping preferences, entertainment preferences, and financial preferences. Shopping preferences include favorite stores, shopping frequency, categories shopped, favorite brands. Entertainment preferences include network/cable/satellite access capabilities, favorite shows, favorite genres, and favorite actors. Financial preferences include favorite insurance companies, preferred investment practices, banking preferences, and favorite online financial instruments. A variety of subject attributes may be included in a subject attributes data model 315 and data models may be preset or custom generated to suit particular purposes.

According to various embodiments, data models for neuro-feedback association 325 identify experimental protocols 327, modalities included 329 such as measurement mechanisms, surveys conducted, and experiment design parameters 333 such as segments and segment attributes. Other fields may include experiment presentation scripts, segment length, segment details like stimulus material used, inter-subject variations, intra-subject variations, instructions, presentation order, survey questions used, etc. Other data models may include a data collection data model 337. According to various embodiments, the data collection data model 337 includes recording attributes 339 such as station and location identifiers, the data and time of recording, and operator details. In particular embodiments, equipment attributes 341 include an amplifier identifier and a sensor identifier.

Modalities recorded 343 may include modality specific attributes like eye tracking specific attributes. Eye tracking specific attributes include the type of tracker used, data recording frequency, data being recorded, recording format, etc. According to various embodiments, data storage attributes 345 include file storage conventions (format, naming convention, dating convention), storage location, archival attributes, expiry attributes, etc.

A preset query data model 349 includes a query name 351 and/or identifier, an accessed data collection 353 such as data segments involved (models, databases/cubes, tables, etc.), access security attributes 355 included who has what type of access, and refresh attributes 357 such as the expiry of the query, refresh frequency, etc. Other fields such as push-pull preferences can also be included to identify an auto push reporting driver or a user driven report retrieval system.

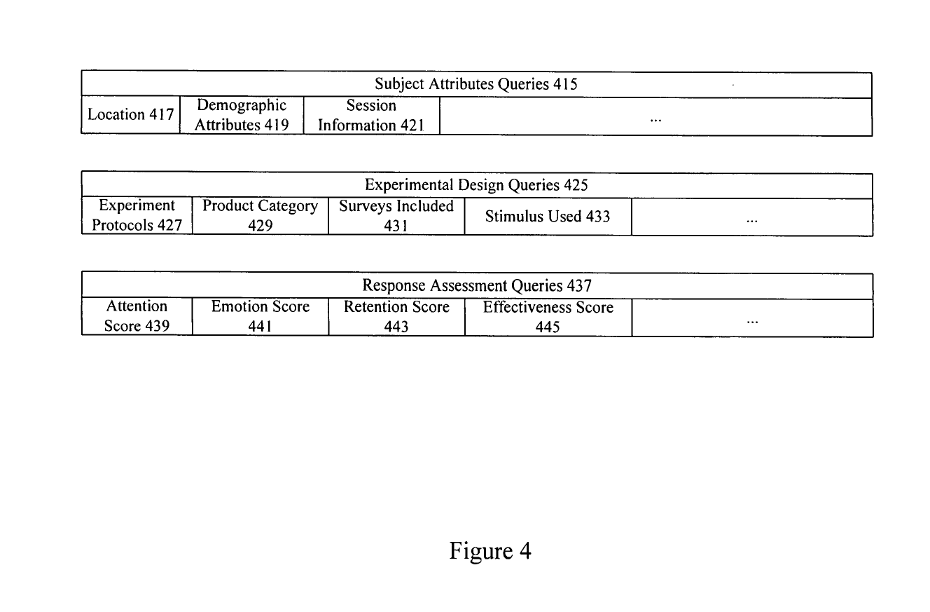

FIG. 4 illustrates examples of queries that can be performed to obtain data associated with response assessment. According to various embodiments, queries are defined from general or customized scripting languages and constructs, visual mechanisms, a library of preset queries, diagnostic querying including drill-down diagnostics, and eliciting what if scenarios. According to various embodiments, subject attributes queries 415 may be configured to obtain data from a neuro-informatics repository using a location 417 or geographic information, session information 421 such as testing times and dates, and demographic attributes 419. Demographics attributes include household income, household size and status, education level, age of kids, etc.

Other queries may retrieve stimulus material based on shopping preferences of subject participants, countenance, physiological assessment, completion status. For example, a user may query for data associated with product categories, products shopped, shops frequented, subject eye correction status, color blindness, subject state, signal strength of measured responses, alpha frequency band ringers, muscle movement assessments, segments completed, etc. Experimental design based queries may obtain data from a neuro-informatics repository based on experiment protocols 427, product category 429, surveys included 431, and stimulus provided 433. Other fields that may used include the number of protocol repetitions used, combination of protocols used, and usage configuration of surveys.

Client and industry based queries may obtain data based on the types of industries included in testing, specific categories tested, client companies involved, and brands being tested. Response assessment based queries 437 may include attention scores 439, emotion scores, 441, retention scores 443, and effectiveness scores 445. Such queries may obtain materials that elicited particular scores.

Response measure profile based queries may use mean measure thresholds, variance measures, number of peaks detected, etc. Group response queries may include group statistics like mean, variance, kurtosis, p-value, etc., group size, and outlier assessment measures. Still other queries may involve testing attributes like test location, time period, test repetition count, test station, and test operator fields. A variety of types and combinations of types of queries can be used to efficiently extract data.

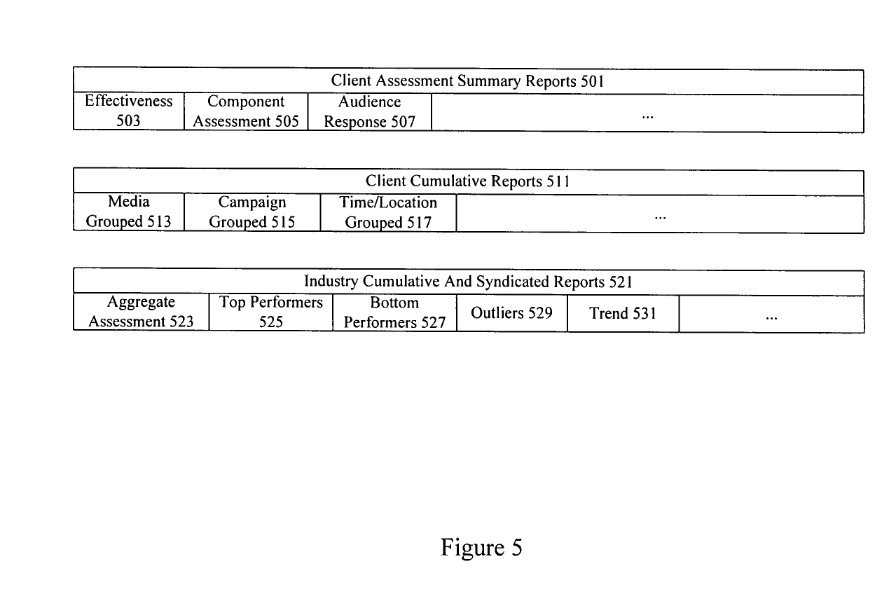

FIG. 5 illustrates examples of reports that can be generated. According to various embodiments, client assessment summary reports 501 include effectiveness measures 503, component assessment measures 505, and response measures 507. Effectiveness assessment measures include composite assessment measure(s), industry/category/client specific placement (percentile, ranking, . . . ), actionable grouping assessment such as removing material, modifying segments, or fine tuning specific elements, etc, and the evolution of the effectiveness profile over time. In particular embodiments, component assessment reports include component assessment measures like attention, emotional engagement scores, percentile placement, ranking, etc. Component profile measures include time based evolution of the component measures and profile statistical assessments. According to various embodiments, reports include the number of times material is assessed, attributes of the multiple presentations used, evolution of the response assessment measures over the multiple presentations, and usage recommendations.

According to various embodiments, client cumulative reports 511 include media grouped reporting 513 of all stimulus assessed, campaign grouped reporting 515 of stimulus assessed, and time/location grouped reporting 517 of stimulus assessed. According to various embodiments, industry cumulative and syndicated reports 521 include aggregate assessment responses measures 523, top performer lists 525, bottom performer lists 527, outliers 529, and trend reporting 531. In particular embodiments, tracking and reporting includes specific products, categories, companies, brands.

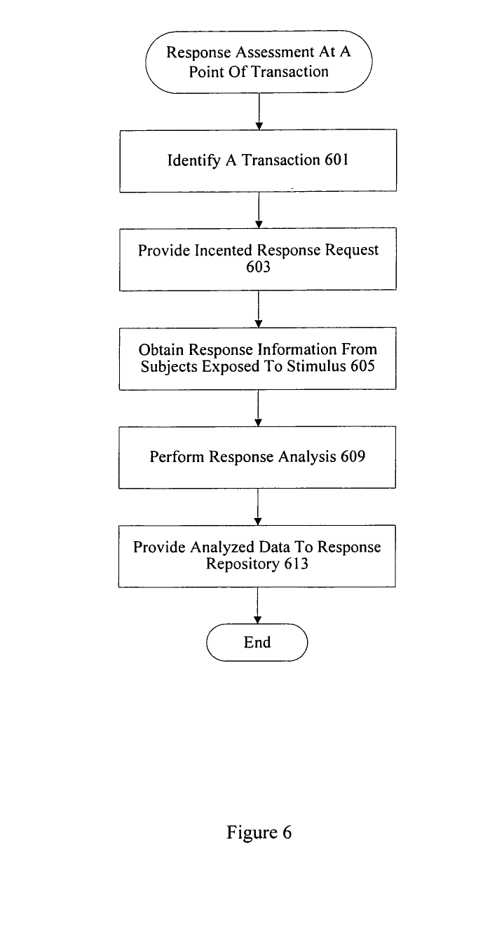

FIG. 6 illustrates one example of response assessment at a point of transaction. At 601, a transaction is identified. According to various embodiments, any point of transaction such as a product request, service request, data request, product delivery, service fulfillment, data download, etc. can be identified. At 603, an incented response request is made to present an incentive and request user response. According to various embodiments, the incentive is associated with the transaction such as a product, service, and/or data request. In particular embodiments, a request is made at the point of transaction for user responses to stimulus. The stimulus may be obtained from a stimulus and audience attributes repository and may use information about user and/or user group profiles. At 605, response data is obtained from subjects exposed to stimulus. According to various embodiments, stimulus includes streaming video, media clips, printed materials, presentations, performances, games, etc. Stimulus presentation may also intelligently use protocols that determine parameters surrounding the presentation of stimulus, such as the number of times shown, the duration of the exposure, sequence of exposure, segments of the stimulus to be shown, etc. According to various embodiments, responses are collected using a variety of mechanisms such as questionnaires, surveys, switches. In some examples, neuro-response collection mechanisms such as EEG, ERP, EOG, GSR, eye-tracking, etc., can also be used. In some examples, verbal and written responses are collected and correlated with behavioral, statistical, survey, and neurophysiological responses.

The data may be passed to a data cleanser to remove noise and artifacts that may make data more difficult to interpret.

At 609, response analysis is performed. Response analysis may include analysis of subject verbal and written responses, as well as analysis of neuro-response measures. In particular embodiments, EEG response data is synthesized to provide an enhanced assessment of effectiveness. According to various embodiments, EEG measures electrical activity resulting from thousands of simultaneous neural processes associated with different portions of the brain. EEG data can be classified in various bands. According to various embodiments, brainwave frequencies include delta, theta, alpha, beta, and gamma frequency ranges. Delta waves are classified as those less than 4 Hz and are prominent during deep sleep. Theta waves have frequencies between 3.5 to 7.5 Hz and are associated with memories, attention, emotions, and sensations. Theta waves are typically prominent during states of internal focus.

Alpha frequencies reside between 7.5 and 13 Hz and typically peak around 10 Hz. Alpha waves are prominent during states of relaxation. Beta waves have a frequency range between 14 and 30 Hz. Beta waves are prominent during states of motor control, long range synchronization between brain areas, analytical problem solving, judgment, and decision making. Gamma waves occur between 30 and 60 Hz and are involved in binding of different populations of neurons together into a network for the purpose of carrying out a certain cognitive or motor function, as well as in attention and memory. Because the skull and dermal layers attenuate waves in this frequency range, brain waves above 75-80 Hz are difficult to detect and are often not used for stimuli response assessment.

However, the techniques and mechanisms of the present invention recognize that analyzing high gamma band (kappa-band: Above 60 Hz) measurements, in addition to theta, alpha, beta, and low gamma band measurements, enhances neurological attention, emotional engagement and retention component estimates. In particular embodiments, EEG measurements including difficult to detect high gamma or kappa band measurements are obtained, enhanced, and evaluated. Subject and task specific signature sub-bands in the theta, alpha, beta, gamma and kappa bands are identified to provide enhanced response estimates. According to various embodiments, high gamma waves (kappa-band) above 80 Hz (typically detectable with sub-cranial EEG and/or magnetoencephalography) can be used in inverse model-based enhancement of the frequency responses to the stimuli.

Various embodiments of the present invention recognize that particular sub-bands within each frequency range have particular prominence during certain activities. A subset of the frequencies in a particular band is referred to herein as a sub-band. For example, a sub-band may include the 40-45 Hz range within the gamma band. In particular embodiments, multiple sub-bands within the different bands are selected while remaining frequencies are band pass filtered. In particular embodiments, multiple sub-band responses may be enhanced, while the remaining frequency responses may be attenuated.

An information theory based band-weighting model is used for adaptive extraction of selective dataset specific, subject specific, task specific bands to enhance the effectiveness measure. Adaptive extraction may be performed using fuzzy scaling. Stimuli can be presented and enhanced measurements determined multiple times to determine the variation profiles across multiple presentations. Determining various profiles provides an enhanced assessment of the primary responses as well as the longevity (wear-out) of the marketing and entertainment stimuli. The synchronous response of multiple individuals to stimuli presented in concert is measured to determine an enhanced across subject synchrony measure of effectiveness. According to various embodiments, the synchronous response may be determined for multiple subjects residing in separate locations or for multiple subjects residing in the same location.

Although a variety of synthesis mechanisms are described, it should be recognized that any number of mechanisms can be applied—in sequence or in parallel with or without interaction between the mechanisms.

Although intra-modality synthesis mechanisms provide enhanced significance data, additional cross-modality synthesis mechanisms can also be applied. A variety of mechanisms such as EEG, Eye Tracking, GSR, EOG, and facial emotion encoding are connected to a cross-modality synthesis mechanism. Other mechanisms as well as variations and enhancements on existing mechanisms may also be included. According to various embodiments, data from a specific modality can be enhanced using data from one or more other modalities. In particular embodiments, EEG typically makes frequency measurements in different bands like alpha, beta and gamma to provide estimates of significance. However, the techniques of the present invention recognize that significance measures can be enhanced further using information from other modalities.

For example, facial emotion encoding measures can be used to enhance the valence of the EEG emotional engagement measure. EOG and eye tracking saccadic measures of object entities can be used to enhance the EEG estimates of significance including but not limited to attention, emotional engagement, and memory retention. According to various embodiments, a cross-modality synthesis mechanism performs time and phase shifting of data to allow data from different modalities to align. In some examples, it is recognized that an EEG response will often occur hundreds of milliseconds before a facial emotion measurement changes. Correlations can be drawn and time and phase shifts made on an individual as well as a group basis. In other examples, saccadic eye movements may be determined as occurring before and after particular EEG responses. According to various embodiments, time corrected GSR measures are used to scale and enhance the EEG estimates of significance including attention, emotional engagement and memory retention measures.

Evidence of the occurrence or non-occurrence of specific time domain difference event-related potential components (like the DERP) in specific regions correlates with subject responsiveness to specific stimulus. According to various embodiments, ERP measures are enhanced using EEG time-frequency measures (ERPSP) in response to the presentation of the marketing and entertainment stimuli. Specific portions are extracted and isolated to identify ERP, DERP and ERPSP analyses to perform. In particular embodiments, an EEG frequency estimation of attention, emotion and memory retention (ERPSP) is used as a co-factor in enhancing the ERP, DERP and time-domain response analysis.

EOG measures saccades to determine the presence of attention to specific objects of stimulus. Eye tracking measures the subject's gaze path, location and dwell on specific objects of stimulus. According to various embodiments, EOG and eye tracking is enhanced by measuring the presence of lambda waves (a neurophysiological index of saccade effectiveness) in the ongoing EEG in the occipital and extra striate regions, triggered by the slope of saccade-onset to estimate the significance of the EOG and eye tracking measures. In particular embodiments, specific EEG signatures of activity such as slow potential shifts and measures of coherence in time-frequency responses at the Frontal Eye Field (FEF) regions that preceded saccade-onset are measured to enhance the effectiveness of the saccadic activity data.

GSR typically measures the change in general arousal in response to stimulus presented. According to various embodiments, GSR is enhanced by correlating EEG/ERP responses and the GSR measurement to get an enhanced estimate of subject engagement. The GSR latency baselines are used in constructing a time-corrected GSR response to the stimulus. The time-corrected GSR response is co-factored with the EEG measures to enhance GSR significance measures.

According to various embodiments, facial emotion encoding uses templates generated by measuring facial muscle positions and movements of individuals expressing various emotions prior to the testing session. These individual specific facial emotion encoding templates are matched with the individual responses to identify subject emotional response. In particular embodiments, these facial emotion encoding measurements are enhanced by evaluating inter-hemispherical asymmetries in EEG responses in specific frequency bands and measuring frequency band interactions. The techniques of the present invention recognize that not only are particular frequency bands significant in EEG responses, but particular frequency bands used for communication between particular areas of the brain are significant. Consequently, these EEG responses enhance the EMG, graphic and video based facial emotion identification.

At 613, processed data is provided to a response repository for querying, processing, report generation, etc. According to various embodiments, the response repository combines analyzed and enhanced responses to the stimulus material while using information about stimulus material attributes. In particular embodiments, the response repository also collects and integrates user behavioral and survey responses with the analyzed and enhanced response data to more effectively measure and track response to stimulus materials. According to various embodiments, the response repository obtains attributes such as requirements and purposes of the stimulus material presented.

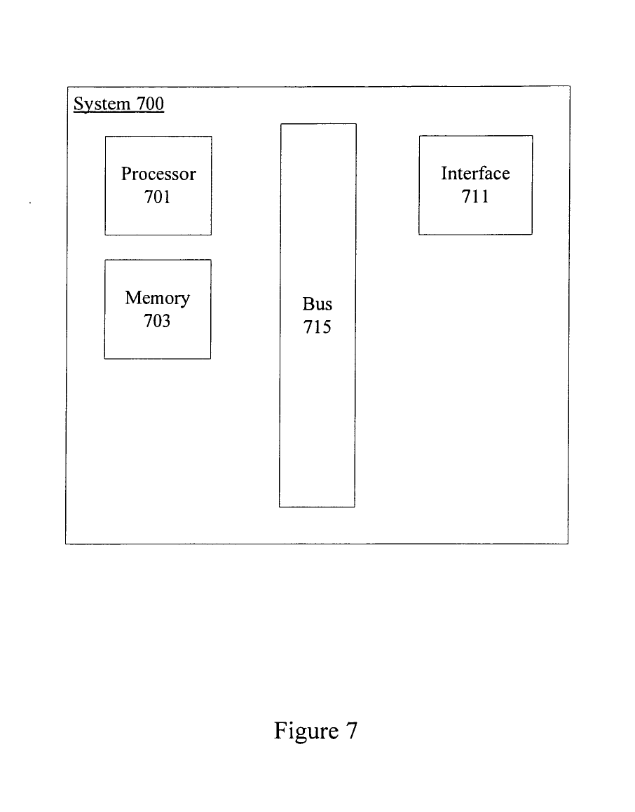

According to various embodiments, various mechanisms such as the data collection mechanisms, the intra-modality synthesis mechanisms, cross-modality synthesis mechanisms, etc. are implemented on multiple devices. However, it is also possible that the various mechanisms be implemented in hardware, firmware, and/or software in a single system. FIG. 7 provides one example of a system that can be used to implement one or more mechanisms. For example, the system shown in FIG. 7 may be used to implement a response analyzer.

According to particular example embodiments, a system 700 suitable for implementing particular embodiments of the present invention includes a processor 701, a memory 703, an interface 711, and a bus 715 (e.g., a PCI bus). When acting under the control of appropriate software or firmware, the processor 701 is responsible for such tasks such as pattern generation. Various specially configured devices can also be used in place of a processor 701 or in addition to processor 701. The complete implementation can also be done in custom hardware. The interface 711 is typically configured to send and receive data packets or data segments over a network. Particular examples of interfaces the device supports include host bus adapter (HBA) interfaces, Ethernet interfaces, frame relay interfaces, cable interfaces, DSL interfaces, token ring interfaces, and the like.

In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control such communications intensive tasks as data synthesis.

According to particular example embodiments, the system 700 uses memory 703 to store data, algorithms and program instructions. The program instructions may control the operation of an operating system and/or one or more applications, for example. The memory or memories may also be configured to store received data and process received data.

Because such information and program instructions may be employed to implement the systems/methods described herein, the present invention relates to tangible, machine readable media that include program instructions, state information, etc. for performing various operations described herein. Examples of machine-readable media include, but are not limited to, magnetic media such as hard disks, floppy disks, and magnetic tape; optical media such as CD-ROM disks and DVDs; magneto-optical media such as optical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

Although the foregoing invention has been described in some detail for purposes of clarity of understanding, it will be apparent that certain changes and modifications may be practiced within the scope of the appended claims. Therefore, the present embodiments are to be considered as illustrative and not restrictive and the invention is not to be limited to the details given herein, but may be modified within the scope and equivalents of the appended claims.

What is claimed is:

1. A system, comprising:
a transaction identifier operable to identify a transaction and a subject participating in the transaction;
an incented response request device operable to present an incentive at a point of transaction for the subject to provide response data;
a response collection device operable to obtain response data from the subject presented with the incentive, wherein the subject is exposed to stimulus material obtained from a stimulus repository;
a response analyzer operable to receive response data from the subject and process the response data to generate enhanced response data.
a response repository operable to maintain enhanced response data to allow assessment of the effectiveness of the stimulus material at the point of transaction.

2. The system of claim 1, wherein the stimulus repository is a stimulus and audience attribute repository.

3. The system of claim 1, wherein the response data includes survey data.

4. The system of claim 1, wherein the response data includes verbal and written data.

5. The system of claim 1, wherein the response data includes neuro-response data.

6. The system of claim 5, wherein neuro-response data includes central nervous system and autonomic nervous system data.

7. The system of claim 5, wherein neuro-response data includes central nervous system and effector data.

8. The system of claim 1, wherein behavioral, statistical, survey, and neurophysiological measurements including attention, emotion, and memory retention are used to analyze response data.

9. The system of claim 1, wherein the stimulus material is marketing or entertainment material

10. The system of claim 1, wherein the transaction is a request for a service.

11. The system of claim 1, wherein the transaction is a request for a product.

12. The system of claim 1, wherein the transaction is a request for data.

13. The system of claim 1, wherein the response collection device includes statistical and survey estimates using nonlinear, geometric, and spiral rating mechanisms.

14. A method, comprising:
identifying a transaction and a subject participating in the transaction;
presenting an incentive at a point of transaction for the subject to provide response data;
obtaining response data from the subject presented with the incentive, wherein the subject is exposed to stimulus material obtained from a stimulus repository;
receiving response data from the subject and processing the response data to generate enhanced response data;
maintaining enhanced response data to allow assessment of the effectiveness of the stimulus material at the point of transaction.

15. The method of claim 14, wherein the stimulus repository is a stimulus and audience attribute repository.

16. The method of claim 14, wherein the response data includes survey data.

17. The method of claim 14, wherein the response data includes verbal and written data.

18. The method of claim 14, wherein the response data includes neuro-response data.

19. The method of claim 18, wherein neuro-response data includes central nervous system and autonomic nervous system data.

20. The method of claim 18, wherein neuro-response data includes central nervous system and effector data.

21. The method of claim 14, wherein behavioral, statistical, survey, and neurophysiological measurements including attention, emotion, and memory retention are used to analyze response data.

22. The method of claim 14, wherein the stimulus material is marketing or entertainment material

23. The method of claim 14, wherein the transaction is a request for a service.

24. The method of claim 14, wherein the transaction is a request for a product.

25. The system of claim 14, wherein the transaction is a request for data.

26. An apparatus, comprising:
means for identifying a transaction and a subject participating in the transaction;
means for presenting an incentive at a point of transaction for the subject to provide response data;
means for obtaining response data from the subject presented with the incentive, wherein the subject is exposed to stimulus material obtained from a stimulus repository;
means for receiving response data from the subject and processing the response data to generate enhanced response data;
means for maintaining enhanced response data to allow assessment of the effectiveness of the stimulus material at the point of transaction.
